(19) United States
(12) Patent Application Publication
(10) Pub. No.: US 2020/0206150 A1
(43) Pub. Date: Jul. 2, 2020

(54) CAROTENOID COMPOSITIONS HAVING ANTIVIRAL ACTIVITIES AND USES THEREOF

(71) Applicant: I.B.R. Israeli Biotechnology Research Ltd., Yavne (IL)

(72) Inventors: Eyal Kalo, Ness-Ziona (IL); Fabien Havas, Bnei Dkalim (IL); Liki von Oppen-Bezalel, Berlin (DE); Olga Ben-Chitrit, Ashdod (IL); Inon Perry, Tel Aviv (IL)

(21) Appl. No.: 15/753,627

(22) PCT Filed: Aug. 18, 2016
(86) PCT No.: PCT/IL16/50909

§ 371 Date: Feb. 20, 2018

Related U.S. Application Data
(60) Provisional application No. 62/207,422, filed on Aug. 20, 2015.
(60) Provisional application No. 62/216,409, filed on Sep. 10, 2015.

(51) Int. Cl. A61K 31/01; A61P 31/16; A61P 31/22

(52) U.S. Cl. CPC A61K 31/01; A61P 31/22; A61P 31/16

(57) ABSTRACT

The present invention relates to compositions comprising carotenoids, particularly phytoene and phytofluene, useful in delaying viral infection, particularly via modulation of at least one response against the viral infection within a cell or tissue. The cells or tissue can be of a subject, thereby delaying the viral infection in the subject or isolated, particularly in a form of cell or tissue culture, particularly for use in methods of screening for anti-viral agents.

### FIELD OF THE INVENTION

The present invention relates to compositions comprising carotenoids, particularly phytoene and phytofluene, useful in delaying viral infection in a subject and in methods of screening for anti-viral agents.

### BACKGROUND OF THE INVENTION

A virus is a small infectious agent that replicates only inside living cells of other organisms. Viruses can infect various life forms, from animals and plants to microorganisms, including bacteria and archaea. Virus infection typically causes disease symptoms in the affected subject, which can vary from mild to severe symptoms responsible for morbidity and mortality.

One of the most abandoned symptoms associated with viral infection is human respiratory diseases. Examples of human respiratory diseases caused by viruses include influenza, caused by various influenza viruses, severe acute respiratory syndrome (SARS), caused by coronavirus (SARS-CoV), illness of the respiratory system caused by adenoviruses and bronchitis and children pneumonia caused by the respiratory syncytial virus (RSV).

Many other serious diseases such as Ebola virus disease, AIDS and herpes simplex virus 1 (HSV-1) are caused by viruses. HSV-1 is a virus attacking nerve cells, causing cold sores among other conditions.

Influenza, commonly known as “the flu”, is an infectious disease caused by the influenza viruses. Influenza spreads around the world in a yearly outbreak, resulting in about three to five million cases of severe illness and about 250,000 to 500,000 deaths. Death occurs mostly in the young, the old and those with other health problems. Complications of influenza may include viral pneumonia, secondary bacterial pneumonia, sinus infections, and worsening of previous health problems such as asthma or heart failure.

Typically, the influenza virus is spread through the air from coughs or sneezes. This is believed to occur mostly over relatively short distances. It can also be spread by touching surfaces contaminated by the virus and then touching the mouth or eyes. Frequent hand washing and wearing a surgical mask are common preventive methods.

Three main influenza subtypes cause the disease, Influenza A, B and C, which are classified according to their antigenic determinants. The influenza virion consists of a single stranded RNA genome closely associated with a nucleoprotein (NP) and enclosed by a lipoprotein envelope lined by matrix protein (M1) and carrying two major surface glycoprotein antigens, haemagglutinin (HA) and neuraminidase (NA). The HA and NA glycoproteins are most susceptible to change; for example, there are 16 immune classes of HA and 9 different NA classes that provide the basis for the different influenza virus subtypes like H1N1 or H3N2. Influenza A virus has an additional transmembrane glycoprotein, M2, which is highly conserved between the different HN subtypes. The M2 gene encodes a protein having 96-97-amino-acids that is expressed as a tetramer on the virion cell surface. It is composed of about 24 extracellular amino acids, about 19 transmembrane amino acids, and about 54 cytoplasmic residues.

The two classes of antiviral drugs commonly used against influenza are neuraminidase inhibitors (e.g. oseltamivir, marketed under the trade name “Tamiflu” and zanamivir) and M2 protein inhibitors (adamantane derivatives). Research of neuraminidase inhibitors suggests that their benefits in those patients who are otherwise healthy might not be greater than the risks involved in taking the drugs, and in those with other health problems their benefit might be lacking altogether. The antiviral drugs amantadine and rimantadine inhibit a viral ion channel (M2 protein), thus inhibiting replication of the influenza A virus. These drugs are sometimes effective against influenza A if given early in the infection but are ineffective against influenza B viruses, which lack the M2 drug target. Measured resistance to amantadine and rimantadine of American isolates of H3N2 has increased to 91% in 2005. The Centers for Disease Control (CDC) has been known to recommend against using M2 inhibitors due to high levels of drug resistance. Yearly vaccinations against influenza are recommended by the World Health Organization for those at high risk. However, a vaccine made against the abandoned viruses of one year may not be useful in the following year, since the virus evolves rapidly. A safe and effective agent for treatment of influenza disease is presently an unmet need of the health care community and general population.

Carotenoids are a family of over 600 members of naturally occurring organic pigments synthesized by plants, algae, and photosynthetic bacteria. Carotenoids can be produced from fats and other basic organic metabolic building blocks by all these organisms. Humans are not able to produce carotenoids, but they are able to accumulate carotenoids from their diet.

Carotenoids are split into two classes, xanthophylls (which contain oxygen) and carotenes (which are purely hydrocarbons, and contain no oxygen). All carotenoids are tetraterpenoids, meaning that they are produced from 8 isoprene molecules and contain 40 carbon atoms. Carotenoids that contain unsubstituted beta-ionone rings (including beta-carotene, alpha-carotene, beta-cryptoxanthin and gamma-carotene) have vitamin A activity (meaning that they can be converted to retinal).

The non-pro-vitamin A carotenoids, including phytoene and phytofluene, carotenoids from the carotene class, are the precursor carotenoids in the biosynthetic pathway of visibly colored carotenoids, for example lycopene. The phytoene and phytofluene molecules are colorless in the visible range, meaning that they absorb light in the UV range only. The benefits to human health of phytoene and phytofluene as anti-oxidant, anti-inflammatory, UV protecting and skin whitening agents have been previously disclosed by some of the inventors of the present invention and co-workers.

Carotenoids are long recognized for their health-promoting properties, typically attributed to their anti-oxidative and anti-inflammatory effects. Carotenoids have thus been also proposed as anti-viral agents, based on the indirect contribution of the anti-oxidative and anti-inflammatory activities to the function of the immune system and overall strength of the body.

U.S. Patent Application Publication No. 2007/0031356 and US2011/0082218 discloses UV mediated effect of beta-carotene on the expression of pro-inflammatory genes. Lin H-W et al., (2012. Food Chem 134:2169-2175) describe the anti-inflammatory effect of beta-carotene and its potential use as anti-inflammatory agent for DNA virus infection. Wertz K et al., (2005. J Invest Dermatol 124:428-434) and Torma H et al., (2014). Arch Dermatol Res 306:739-747), Balmer J E and Blomhoff R. (2002. J Lipid Res. 43:1773-1808), Bertram J H and Bortkiewicz H. (1995. Am J Clin Nutr 62(suppl):13275-365) and describe retinoic acid mediated cellular gene expression. However, none of these publications had addressed the potential effects of the expressed genes on protection against viral infection.

U.S. Pat. No. 5,514,667 discloses the uses a topical preparation comprising an anti-viral drug in combination with a potentiating drug which is an anti-inflammatory or an anti-oxidant drug for the topical treatment of herpes virus infections of the skin and mucous membranes. Carotenoids are mentioned as possible anti-oxidant potentiating the activity of the drug. A preferred preparation disclosed includes zinc pentosan polysulphate in combination with bufexamac.

German Patent Application Publication No. DE202005007462 discloses medicament or nutritional supplement composition, for combating chronic viral infections, containing vitamin C, vitamin E, coenzyme Q10, carotenoids, selenium, zinc and copper.

U.S. Patent Application Publication No. 2009/0169586 discloses a process for preparing a stable packaged dosage form comprising an oxidation-sensitive material, for example whole dried algae of the genus Dunaliella, which is rich in a mixture of carotenoids considered valuable to human health, including β-carotene, a-carotene, lutein, zeaxanthin and cryptoxanthin, as well as in various minerals and other nutritional constituents. The Application discloses methods of using the dosage form comprising the dried Dunaliella for treatment or prophylaxis of various conditions, including fungal and viral infections. Further disclosed is a method for supplementing the diet of a subject, a method for maintaining or improving the general health of a subject, and a method for promoting a fake suntan on a subject.

U.S. Patent Application Publication No. 20110217244 discloses the use of oxidatively transformed carotenoid or a component thereof to enhance the immune response in a subject for the treatment of infection or to enhance the immune response to an antigen in a subject being immunized. Also disclosed are pharmaceutical compositions and kits containing the oxidatively transformed carotenoid.

There is an unmet need for and it would be highly advantageous to have a composition having anti-viral effects, particularly against influenza viruses, which is effective and safe for use.

### SUMMARY OF THE INVENTION

The present invention discloses the use of a combination of phytoene and phytofluene and compositions comprising same for delaying viral infection.

The present invention is based in part on the unexpected discovery that a combination of the phytoene and phytofluene is highly effective in reducing the number of virus particles, more specifically influenza virus particles, virus particles following incubation in vitro as well as in reducing the virus particle load in vivo. The combination was also shown to have a direct effect the expression of genes involved in the cell and body defense mechanisms against viral attack, thus providing a protection mechanism against the infection. The combination is highly effective in inducing the expression of genes involved in reducing the synthesis of viral RNA, in RNA translation and in protein synthesis, thus delaying the virus replication.

The combination was also shown to reduce the expression of genes encoding motor proteins that facilitate the virus movement within the cell thus reducing viral movements required for maintaining the viral replication and infection.

The combination was also shown to reduce the expression of genes essential to the entry of viruses into host cells, thus reducing further spreading and infection by the viruses.

The combination was also shown to affect the expression of genes involved in hijacking the host cell functions, particularly reducing the expression of genes essential for DNA replication and transcription.

Without wishing to be bound to any specific theory or mechanism of action, the anti-viral effect of the compositions of the present invention may be attributed to the induction of a number of cellular anti-viral mechanisms resulting in reduced viral binding and entry into the host cells; reduced DNA/RNA replication, translation and synthesis; and reduced virus spreading to neighboring host cells. None of the suggested modes of activity have been previously attributed to pro-vitamin A compounds or to other carotenoids.

The unexpected finding that phytoene and phytofluene are effective in delaying viral infections via innate cellular defense mechanism independent on activation of the central immune system response, provide a solution for a long felt need of anti-viral treatments for individuals with compromised or undeveloped immune system, including disease-associated immuno-compromised individuals, pregnant women, children and old people. These populations, being highly susceptible to viral infections and their complications, are in the greatest need for anti-viral therapy, yet may be adversely and\or insufficiently affected by agents acting solely through the central immune system.

Furthermore, the unexpected finding that a combination of phytoene and phytofluene is effective in delaying viral infections via innate cellular defense mechanism independent of the central immune system response, allow its use as analytical agent, particularly in screening for effective anti-viral compounds as a positive control in in-vitro tests.

According to one aspect, the present invention provides a method for delaying viral infection of a cell or tissue of a subject in need thereof, the method comprising administering to the subject an effective amount of a combination of phytoene and phytofluene thereby delaying the viral infection.

According to certain embodiments, delaying the viral infection comprises modulating at least one response against the viral infection within the cell or tissue of the subject.

According to certain embodiments, wherein the response against the viral infection is selected from the group consisting of preventing and/or delaying virus adherence and/or penetration into the subject cell or tissue; inhibiting the virus replication within a cell of the subject comprising the virus; delaying hijacking at least one cell function by the virus in a cell comprising the virus; delaying the spread of the virus from a cell comprising the virus into at least one additional cell; inducing the expression of at least one gene associated with a cellular mechanism suppressing the viral infection; and inhibiting the expression of at least one gene associated with a cellular mechanism inducing the viral infection. Each possibility represents a separate embodiment of the present invention.

As used herein, the term “effective amount” of a combination of phytoene and phytofluene refers to the amount effective in modulating at least one response against the viral infection within the cell or a tissue as described herein.

According to certain embodiments, the effective amount of the combination of phytoene and phytofluene is at least 0.05 μM. According to other embodiments, the effective amount of the combination of phytoene and phytofluene is at least 0.1 μM, at least 0.2 μM, at least 0.3 μM, at least 0.4 μM, at least 0.5 μM, at least 1.0 μM, at least 2.0 μM, at least 4.0 μM, at least 6.0 μM, at least 8.0 μM, or at least 10.0 μM. Each possibility represents a separate embodiment of the present invention. According to certain exemplary embodiments, the effective amount of the combination of phytoene and phytofluene is from about 0.05 μm to about 100 μm. According to additional certain exemplary embodiments, the effective amount of the combination of phytoene and phytofluene is from about 0.2 μm to about 100 μm.

According to certain embodiments, the combination of phytoene and phytofluene is administered within a composition.

According to certain currently exemplary embodiments, the phytoene and phytofluene are in their natural form. The phytoene, phytofluene or a combination thereof can be isolated from a natural source, chemically produced or produced using recombinant methods.

According to certain embodiments, the composition comprising the combination of phytoene and phytofluene further comprises at least one additional ingredient selected from the group consisting of amino acid, peptide, polypeptide, fatty acid, fat, sugar compound, a carotenoid other than phytoene and phytofluene or combination thereof. Carotenoids other than phytoene and phytofluene which may be present in the compositions according to these embodiments are selected from the group consisting of zeta-carotene, beta-carotene, lycopene, zeaxanthin, astaxanthin, lutein, canthaxanthin, neurosporene and the like, including all their stereoisomers, derivatives and any combination thereof.

According to yet other embodiments, the carotenoids other than phytoene and phytofluene are of the xanthophylls group. According to further embodiments, the carotenoids other than phytoene and phytofluene are non-pro-vitamin A carotenoids.

According to some embodiments, the composition is essentially devoid of pro-vitamin A carotenoid compounds. According to these embodiments, the composition comprises less than 20% pro-vitamin A carotenoid compounds out of the total carotenoid content of the composition, less than 15%, less than 10%, less than 5%, less than 4%, less than 3%, less than 2%, less than 1% or below pro-vitamin A carotenoid compounds out of the total carotenoid content of the composition. Each possibility represents a separate embodiment of the present invention. According to some embodiments, the composition comprises non-detectable to 10% pro-vitamin A carotenoid compounds out of the total carotenoid content of the composition.

According to some embodiments, the composition is essentially devoid of beta-carotene. According to these embodiments, the composition comprises less than 20% beta carotene out of the total carotenoid content of the composition, less than 15%, less than 10%, less than 5%, less than 1% or below beta-carotene out of the total carotenoid content of the composition. Each possibility represents a separate embodiment of the present invention. According to some embodiments, the composition comprises non-detectable to 10% beta-carotene out of the total carotenoid content of the composition.

According to certain embodiments, the subject in infected with the virus.

According to some embodiments, delaying viral infection of a cell or tissue of the subject results in delaying the onset of the viral infection.

According to embodiments, delaying viral infection of a cell or tissue of the subject results in at least one of preventing, ameliorating, delaying, inhibiting, reducing the severity and any combination thereof of at least one symptom associated with or resulting from the virus infection.

According to these embodiments, the present invention provides a method for treating viral infection in a subject, comprising administering to the subject a therapeutically effective amount of a combination of phytoene and phytofluene or a of a composition comprising same.

According to certain embodiments, the subject is selected from the group consisting of a human and an animal. According to certain exemplary embodiments, the subject is a human.

Without wishing to be bound by any theory or mechanism of action, the direct effect of the combination of phytoene and phytofluene on cellular mechanisms targeted against viral infection as disclosed herein may provide sufficient protection without activation or with moderate activation of the subject systemic immune system, rendering the combination highly suitable for administration to subjects with suppressed immune system or having a disease associated with immunosuppression.

Thus, according to some embodiments, the subject is a subject having compromised immune system. According to other embodiments, the subject is affected with a disease associated with immunosuppression. According to additional embodiments, the subject is receiving or has been receiving immunosuppression treatment. According to certain exemplary embodiments, the immuno-compromised subject is selected from the group consisting of a child, an elderly and a pregnant woman

According to certain embodiments, the viral infection is an infection by influenza virus or a subtype thereof. According to some embodiments, the viral infection is infection by subtype of influenza A virus. According to other embodiments, the viral infection is infection by subtype of influenza B virus. According to additional embodiments, the viral infection is infection by subtype of influenza C virus.

According to certain exemplary embodiments the influenza virus strain is selected from the group consisting of N1H1 PR8 and H3N2. Each possibility represents a separate embodiment of the present invention.

According to certain embodiments, the method of the present invention is applicable for delaying infection by a plurality of variants of the influenza virus.

According to other currently exemplary embodiments, the virus is herpes simplex virus 1 (HSV-1).

According to yet additional embodiments, the method is applicable for delaying infection of a plurality of virus types.

Any method as is known in the art for administering a composition comprising the combination of phytoene and phytofluene according to the teachings of the present invention can be used according to the teachings of the present invention.

According to certain embodiments, the composition is a pharmaceutical composition further comprising a pharmaceutically acceptable excipient, diluents or carrier.

According to other embodiments, the composition is an edible composition further comprising edible excipient, diluents or carrier. According to some embodiments, the edible composition is nutraceutical composition or a nutritional supplement.

According to some embodiments, the pharmaceutical composition is administered via oral, intranasal, buccal, sublingual, topical, subcutaneous and/or intravenous route. According to other embodiments, the composition is administered by inhalation and/or nebulization. Each possibility represents a separate embodiment of the present invention.

According to yet other embodiments the method of the present invention further comprises administering to the subject an additional agent selected from the group consisting of an antiviral agent, a vaccine, an antibody, an anti-oxidant, a traditional herbal medicine agent, a homeopathic remedy, a composition comprising same and any combination thereof.

According certain embodiment, the composition comprising a combination of phytoene and phytofluene according to the invention and the additional agent are administered in a single composition.

According to other embodiments, the combination of phytoene and phytofluene and the at least one additional agent are administered in separate compositions. According to these embodiments, the compositions can be administered simultaneously, concomitantly, sequentially and/or alternately.

The frequency, amount and duration of administration of the combination of phytoene and phytofluene or a composition comprising same depends, among others, on the subject general health, age, gender, weight and occurrence of viral infection. According to some embodiment, the combination of phytoene and phytofluene or a composition comprising same is to be administered from one to eight times per day. According to some embodiment, the combination of phytoene and phytofluene or a composition comprising same is to be administered prophylactically.

According to additional aspect the present invention provides an effective amount of a combination of phytoene and phytofluene or a composition comprising same for use in delaying viral infection of a cell or a tissue of a subject. The composition and the viruses to be treated are as described hereinabove.

According to a further aspect, the present invention provides a method for delaying viral infection of an isolated cell or tissue, comprising administering to the cell or tissue an effective amount of a combination of phytoene and phytofluene or a composition comprising same. According to certain embodiments, the isolated cell or tissue form part of a cell culture or a tissue culture.

According to some embodiments, delaying viral infection of a cell or tissue of the cultures results in delaying the onset of the viral infection. According to some embodiments, delaying viral infection of a cell or tissue results in modulating at least one response against the viral infection within the cell or the tissue.

According to certain embodiments, the cellular response is selected from the group consisting of preventing and/or delaying virus adherence and/or penetration into the cell or tissue; delaying the virus replication within a cell of the subject comprising the virus; delaying hijacking at least one cell function by the virus in a cell comprising the virus; delaying the spread of the virus from a cell comprising the virus into at least one additional cell; inducing the expression of at least one gene associated with a cellular mechanism suppressing the viral infection; and inhibiting the expression of at least one gene associated with a cellular mechanism inducing the viral infection. Each possibility represents a separate embodiment of the present invention.

According some embodiments, administering the combination of phytoene and phytofluene or a composition comprising same for delaying viral infection of an isolated cell or tissue provides a positive control in determining anti-viral activity of a test compound, wherein the test compound is administered to the same type of cell or tissue under the same experimental conditions. According to some embodiments, the combination of phytoene and phytofluene or a composition comprising same is administered at an amount found to have a therapeutic effect in delaying viral infection. According to other embodiments, the combination of phytoene and phytofluene or a composition comprising same is administered at an amount calibrated for the type of cell or tissue used.

Other objects, features and advantages of the present invention will become clear from the following description and drawings. Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 demonstrates that the combination of phytoene and phytofluene (COMP1) have no cytotoxic effect on MDCK cells.

FIG. 2 shows the inhibitory effect of the combination of phytoene and phytofluene (COMP1) on the density of influenza virus (H1N1 PR8) in MDCK cells.

FIG. 3 shows gensin violet staining of human kidney cells infected with HSV-1, treated or non-treated with COMP1. Acyclovir known as an effective HSV1 drug served as a positive control.

FIG. 4 shows an in vivo inhibitory effect of the combination of phytoene and phytofluene (COMP1) on influenza virus particle number.

### DETAILED DESCRIPTION OF THE INVENTION

### Definitions

As used herein, the term “phytoene” refers to 7,7′,8,8′,11,11′,12,12′-octahydro-psi-psi-carotene, including all its stereoisomers. The synthesis of phytoene is the first committed step in the synthesis of carotenoids in plants. Phytoene is produced from two molecules of geranylgeranyl pyrophosphate (GGPP) by the action of the enzyme phytoene synthase. Phytoene is a non pro-vitamin A carotene.

The term “phytofluene” refers to 15-cis, 7, 8, 11, 12, 7′, 8′-hexahydro- psi-psi-carotene including all its stereoisomers and derivatives. Phytofluene is formed from phytoene in a desaturation reaction leading to the formation of five conjugated double bonds. Phytofluene is a non pro-vitamin A carotene.

The molar amounts of phytoene and phytofluene as disclosed herein are measured spectrophotometerically using the following equation:

O.D*dilution factor (=concentration in mg/ml)/Phytoene/Phytofluene coefficient, wherein

Phytoene extinction coefficient is 915 in hexane and ethanol and 870 in ethyl acetate; and

Phytofluene extinction coefficient is 1557 in hexane and ethanol and 1480 in ethyl acetate; and wherein the O.D for phytoene is taken at 284-288 nm and for phytofluene at 345-248 nm.

As used herein, the terms “delay” or “delayed” with reference to viral infection are meant to be used in their broadest scope as to postpone, suspend, hold or completely inhibit all activities and mechanisms employed by a virus for survival, replication and spreading as to cause cellular infection.

As used herein, the term “effective amount” means an amount of a combination of phytoene and phytofluene sufficient to delay viral infection of a cell or a tissue, either in vivo within a subject or in vitro in a cell or tissue culture. According to certain embodiments of the invention, delaying viral infection of a cell or a tissue encompasses modulation of at least one cellular response against the virus, resulting in at least one of reduced invasion, replication and spread of the virus.

As used herein, the term “therapeutically effective amount” refers to an effective amount of a combination of phytoene and phytofluene as described hereinabove that is further effective in treating a subject affected with or suspected to be affected with or prone to be affected with the virus. As used herein, the term “treating” refers to delaying the onset of viral infection and/or preventing, ameliorating, delaying, reducing the severity and any combination thereof of at least one symptom associated with or resulting from the virus infection.

According to one aspect, the present invention provides a method of delaying viral infection of a cell or tissue of a subject in need thereof, comprising administering to the subject a composition comprising an effective amount of a combination of phytoene and phytofluene thereby delaying or inhibiting the viral infection.

According to another aspect, the present invention provides a method of inhibiting viral infection of a cell or tissue of a subject in need thereof, comprising administering to the subject a composition comprising an effective amount of a combination of phytoene and phytofluene thereby delaying or inhibiting the viral infection.

According to certain embodiments, delaying the viral infection comprises modulating at least one response against the viral infection within the cell or tissue of the subject.

According to some embodiments, delaying the viral infection comprises preventing and/or inhibiting virus adherence and/or penetration into the cell or tissue.

According to some embodiments, delaying the viral infection comprises inhibiting the virus replication within a cell of the subject comprising the virus.

According to some embodiments, delaying the viral infection comprises inhibiting the spread of the virus from a cell comprising the virus into at least one additional cell.

According to some embodiments, delaying the viral infection comprises inhibiting hijacking at least one cell function by the virus in a cell comprising the virus.

According to some embodiments, delaying the viral infection comprises inducing the expression of at least one gene associated with a cellular mechanism suppressing the viral infection. According to these embodiments, the at least one gene associated with the cellular mechanism suppressing the viral infection is selected from the group consisting of RSAD2 (NM_080657); IFIT1 (NM_001548) and IFIT3 (NM_001549) and any combination thereof. Each possibility represents a separate embodiment of the present invention.

According to some embodiments, delaying the viral infection comprises inhibiting the expression of at least one gene associated with a cellular mechanism promoting the viral infection. According to these embodiments, the at least one gene associated with the cellular mechanism promoting the viral infection is selected from the group consisting of DDX17 (NM_006386); ATF6 (NM_007348); TOP1 (NM_003286); USP47 (NM_017944); KIF5B (NM_004521); HTATSF1 (NM_001163280); SRPK1 (NM_003137); PCBP2 (NM_005016); ACBD3 (NM_022735) and any combination thereof. Each possibility represents a separate embodiment of the present invention.

Phytoene and phytofluene for use in the methods and compositions of the present invention can be obtained from any available source. They can be extracted from a natural source, can be synthesized chemically or can be obtained by methods of molecular genetics. According to certain exemplary embodiments, phytoene and phytofluene may be obtained from organisms that produce carotenoids, including a variety of plants, various algae, and certain microorganisms, including genetically modified organisms. Non limiting examples include tomato fruit, citrus fruit, carrot, algae, fungi and edible flowers. Each of the phytoene and phytofluene may be extracted from a different source, or may be obtained from the same source.

According to certain embodiments, phytoene, phytofluene or any combination thereof is derived from a tomato fruit. According to other embodiments, phytoene, phytofluene or any combination thereof is derived from an alga, preferably an alga of the species Dunaliella.

According to other embodiments, phytoene and phytofluene are not extracted and/or alternatively isolated or purified from the organism. Thus, according to certain embodiments, the combination of phytoene and phytofluene of the present invention are in the form of powder, obtained by drying the intact organism or a part thereof as to obtain phytoene and phytofluene rich powder. Organisms or parts thereof that can be dried to provide the phytoene and phytofluene combination of the present invention include, for example, algae, fungi, microorganisms, plants and plant parts such as leaves, fruits, stems, roots and flowers.

According to certain embodiments, the phytoene and phytofluene rich powder is further purified. According to some embodiments, the purification is performed by column chromatography or extraction of impurities by supercritical carbon dioxide (SCCO₂).

In addition to obtaining phytoene, phytofluene or the combination thereof from a natural source, these carotenoids can also be synthesized by any of the known chemical or biochemical methods or by recovering these carotenoids from a genetically modified source. Chemically, phytoene can be synthesized, for example, from two geranylgeranyl pyrophosphates (C-20), in a reaction which may be mediated by phytoene synthase. The geranylgeranyl pyrophosphate can be obtained directly, by the conversion of mevalonic acid or by the condensation of pyruvate and glyceraldehyde-3-phosphate. Phytofluene can be synthesized by desaturation of phytoene, a reaction which may be mediated by phytoene desaturase.

A genetically modified source can also be a source for phytoene and phytofluene. The genetically modified surface can be obtained, for example, by the mutagenesis of enzymes which are active downstream to phytofluene in a carotenoid-producing organism. Such synthesized phytoene, phytofluene and other carotenoid will have activities that are substantively similar to the activities of these carotenoids obtained from organisms that produce carotenoids as described hereinabove.

The carotenoid concentration in a composition can be measured by any method as is known to a person skilled in the art. Typically, carotenoid concentration is measured either by high-performance liquid chromatography (HPLC) methods or by spectrophotometer.

The absorption spectra of carotenoids are markedly solvent dependent, and the absorbance coefficient of a carotenoid (absorbance at given wavelength of a 1% solution in spectrophotometric cuvette with a 1-cm light path), which is used in the calculation of the concentration of a certain carotenoid varies significantly in different solvents. This has to be remembered in analyzing carotenoids in a certain composition, particularly when analysis is performed by HPLC in which different solvents may be used during the separation procedure.

The molar amounts of the combination of phytoene and phytofluene disclosed in the present invention are measured as described hereinabove.

Carotenoid extraction from the organism or any of its parts, particularly extraction of phytoene and phytofluene can be performed by any method as is known to a person skilled in that art. According to certain embodiments, the organism or its part is immersed in a solvent, to extract oil soluble ingredients from the organism into the solvent. Extraction may be carried out after applying a pre-treatment. For example, when the source is a tomato fruit, the pre-treatment includes grinding and/or homogenizing the fruit. The extraction process may be accelerated by heating and/or stirring the source-solvent mixture. After removing non-extracted debris, the solvent may be removed. Examples of extract solvents include lower mono alcohols such as methyl alcohol, ethyl alcohol, 1-propanol, 2-propanol, 1-butanol and 2-butanol; lower alkyl esters such as ethyl acetate; hydrocarbons such as benzene, hexane and pentane; ketones such as acetone and methyl ethyl ketone; oils such as squalane, liquid paraffin, polydecene; ethers such as diethyl ether, tetrahydrofuran and dipropyl ether; and acetonitrile. One solvent as well as different solvent combination may be used, as is known to a person skilled in the art. According to certain embodiments, the solvent is hexane, ethanol, ethyl acetate or any combination thereof.

The extract can be then further purified, for example by molecular weight fractionation, solvent fractionation, by purifying the oil soluble extract with any medium such as an ion-exchange resin or an absorbent medium, supercritical CO₂ and the like. According to certain embodiments, the extraction solvent is removed, and the oil-soluble carotenoid preparation is mixed with a carrier suitable for cosmetics or for oral consumption. Such carrier can be a liquid to obtain a liquid formulation or a solid to obtain solid formulation. An example for phytoene and phytofluene extraction is described in U.S. Pat. No. 6,383,474 to some of the inventors of the present invention and co-workers.

According to certain embodiments, the combination of phytoene and phytofluene is administered within a composition.

According to some embodiments, the composition comprising the combination of phytoene and phytofluene is formulated to be water soluble.

A composition comprising a combination of phytoene and phytofluene may further comprise additional compound selected from the group consisting of at least one fatty acid, at least one amino acid, at least one peptide, at least one polypeptide, at least one sugar compound, at least one carotenoid other than phytoene and phytofluene, at least one lipid compound (including phospholipids and fat), at least one glycoprotein, at least one glycoside, at least one alkaloid, at least one terpene compound, at least one phenol compound, at least one polyketide or a combination of any of the above. According to additional embodiments, the composition and at least one fatty acid, at least one amino acid, at least one peptide, at least one polypeptide, at least one sugar compound, at least one carotenoid other than phytoene and phytofluene, at least one lipid compound (including phospholipids and fat), at least one glycoprotein, at least one glycoside, at least one alkaloid, at least one terpene compound, at least one phenol compound, at least one polyketide or a combination of any of the above are provided concomitantly and/or alternately.

The fatty acids, amino acids and sugar compounds which may be present in the compositions according to this embodiment, may be obtained from a natural or GMO source or alternatively, chemically synthesized.

According to yet other embodiments, the carotenoids other than phytoene and phytofluene are of the xanthophylls group. According to further embodiments, the carotenoids other than phytoene and phytofluene are non-pro-vitamin A carotenoids.

According to some embodiments, the composition is essentially devoid of pro-vitamin A carotenoid compounds. According to these embodiments, the composition comprises less than 20% pro-vitamin A carotenoid compounds out of the total carotenoid content of the composition, less than 15%, less than 10%, less than 5%, less than 4%, less than 3%, less than 2%, less than 1% or below pro-vitamin A carotenoid compounds out of the total carotenoid content of the composition. Each possibility represents a separate embodiment of the present invention. According to some embodiments, the composition comprises non-detectable to 10% pro-vitamin A carotenoid compounds out of the total carotenoid content of the composition.

According to some embodiments, the composition is essentially devoid of beta-carotene. According to these embodiments, the composition comprises less than 20% beta carotene out of the total carotenoid content of the composition, less than 15%, less than 10%, less than 5%, less than 1% or below beta-carotene out of the total carotenoid content of the composition. Each possibility represents a separate embodiment of the present invention. According to some embodiments, the composition comprises non-detectable to 10% beta-carotene out of the total carotenoid content of the composition.

According to some embodiments, the content of the combination of phytoene and phytofluene is at least 50%, at least 55%, at least 60%, at least 70%, at least 75%, at least 80%, at least 85%, at least 90%, at least 95% or more out of the total carotenoids content of the composition. Each possibility represents a separate embodiment of the present invention. According to some embodiments, the content of the combination of phytoene and phytofluene is from about 80% to 100% of the total carotenoid content of the composition. According to other embodiments, the only carotenoids present in the composition are phytoene and phytofluene (100% of the total carotenoid content).

According to certain embodiment, the composition comprising the combination of phytoene and phytofluene is devoid of resveratrol. Additionally or alternatively, the composition comprising the combination of phytoene and phytofluene is devoid of non-steroidal anti-inflammatory drugs (NSADs).

According to certain embodiments, the composition comprising the combination of phytoene and phytofluene as described in embodiments of the present invention is a pharmaceutical composition further comprising pharmaceutically acceptable excipients, diluents, carriers or additives. According to these embodiments, the pharmaceutical composition comprises therapeutically effective amount of the combination of phytoene and phytofluene.

One of ordinary skill in the art will recognize that a therapeutically effective amount will vary with the infection or condition to be treated, its severity, the treatment regimen to be employed, the pharmacokinetics of the agent used, as well as the patient (animal or human) treated.

Pharmaceutical compositions comprising a combination of phytoene and phytofluene can be formulated in various dosage forms for oral, nasal, parenteral, intravenous, intramuscular, transdermal, buccal, subcutaneous, suppository and topical administration. In general, it is preferable to administer the pharmaceutical composition in oral-, nasal or topical-administrable form. According to some embodiments, the composition is administered by inhalation. A person skilled in the art may modify the formulations within the teachings of the specification to provide numerous formulations for a particular route of administration without rendering the compositions of the present invention unstable or compromising their therapeutic activity.

To prepare the pharmaceutical compositions according to the present invention, an effective amount of the combination of phytoene and phytofluene according to the present invention is preferably intimately admixed with a pharmaceutically acceptable carrier according to conventional pharmaceutical compounding techniques to produce a dose. A carrier may take a wide variety of forms depending on the form of preparation desired for administration, e.g., oral or parenteral. In preparing pharmaceutical compositions in oral dosage form, any of the usual pharmaceutical media may be used. Thus, for liquid oral preparations such as suspensions, elixirs and solutions, suitable carriers and additives including water, glycols, oils, alcohols, flavoring agents, preservatives, coloring agents and the like may be used. For solid oral preparations such as powders, tablets, capsules, and for solid preparations such as suppositories, suitable carriers and additives including starches, sugar carriers, such as dextrose, mannitol, lactose and related carriers, diluents, granulating agents, lubricants, binders, disintegrating agents and the like may be used. If desired, the tablets or capsules may be enteric-coated or sustained release by standard techniques. The use of these dosage forms may significantly elevate the bioavailability of the phytoene and phytofluene in the patient.

For parenteral formulations, the carrier will usually comprise sterile water or aqueous sodium chloride solution, though other ingredients, including those which aid dispersion, also may be included. Of course, where sterile water is to be used and maintained as sterile, the compositions and carriers must also be sterilized. Injectable suspensions may also be prepared, in which case appropriate liquid carriers, suspending agents and the like may be employed. Liposomal suspensions may also be prepared by conventional methods to produce pharmaceutically acceptable carriers.

The pharmaceutical compositions may also be formulated as modified release dosage forms, including delayed-, extended-, prolonged-, sustained-, pulsed-, controlled-, accelerated- and fast-, targeted-, programmed-release, and gastric retention dosage forms. These dosage forms can be prepared according to conventional methods and techniques known to those skilled in the art (see, e.g., Remington, The Science and Practice of Pharmacy, 21st Edition; Lippincott Williams & Wilkins: Philadelphia, Pa., 2005; Modified-Release Drug Delivery Technology, Rathbone et al., eds., Drugs and the Pharmaceutical Science, Marcel Dekker, Inc.: New York, N.Y., 2003; Vol. 126).

According to certain embodiments, the pharmaceutical compositions are provided in a dosage form for oral administration. According to other embodiments, the pharmaceutical compositions are provided in a dosage form for parenteral administration. According to yet other embodiments, the pharmaceutical compositions are provided in a dosage form for topical administration. According to additional embodiments, the pharmaceutical compositions are provided in a dosage form for intra-nasal administration. According to yet further embodiments, the pharmaceutical compositions are provided in a dosage form as an inhalant.

According to certain embodiments, the pharmaceutical compositions provided herein may be provided in a unit-dosage form or multiple-dosage form. A unit-dosage form, as used herein, refers to a physically discrete unit suitable for administration to human and animal subjects, and packaged individually as is known in the art. Each unit-dose contains a predetermined quantity of the active ingredient(s), a combination of phytoene and phytofluene according to the present invention, sufficient to produce the desired therapeutic effect, in association with the required pharmaceutical carriers or excipients. Examples of a unit-dosage form include an ampoule, syringe, and individually packaged tablet and capsule. A unit-dosage form may be administered in fractions or multiples thereof. A multiple-dosage form is a plurality of identical unit-dosage forms packaged in a single container to be administered in segregated unit-dosage form. Examples of a multiple-dosage form include a vial, bottle of tablets or capsules, or bottle of pre-determined amount of liquid.

The amount of the combination of phytoene and phytofluene included within therapeutically active formulations according to the present invention is an effective amount for delaying the infection and/or affecting at least one cellular mechanism and/or response associated with the viral infection within the cell or tissue of the subject. According to certain exemplary embodiments, the effective amount delays the infection caused by an influenza virus, or in other embodiments, herpes simplex virus 1 (HSV-1). According to yet additional embodiments, the effective amount treat, prevent or delay the onset of influenza or HDV-1 infections in a subject.

According to certain embodiments, the effective amount of the combination of phytoene and phytofluene is at least 0.05 μM. According to other embodiments, the effective amount of the combination of phytoene and phytofluene is at least 0.1 μM, at least 0.2 μM, at least 0.3 μM, at least 0.4 μM, at least 0.5 μM, at least 1.0 μM, at least 2.0 μM, at least 4.0 μM, at least 6.0 μM, at least 8.0 μM, or at least 10.0 μM. Each possibility represents a separate embodiment of the present invention. According to certain exemplary embodiments, the effective amount of the combination of phytoene and phytofluene is from about 0.05 μm to about 100 μm. According to additional certain exemplary embodiments, the effective amount of the combination of phytoene and phytofluene is from about 0.2 μm to about 100 μm.

Administration of the active phytoene and phytofluene combination or a composition comprising same may range from continuous (intravenous drip) to several oral administrations per day (for example, for times a day) and may include oral, topical, parenteral, intramuscular, intravenous, sub-cutaneous, transdermal (which may include a penetration enhancement agent), buccal and suppository administration, among other routes of administration. Enteric coated oral tablets may also be used to enhance bioavailability of the compounds from an oral route of administration. The most effective dosage form will depend upon the severity of disease in the patient and the patient general health, age gender and weight. Oral dosage forms are particularly preferred, because of ease of administration and prospective favorable patient compliance.

The compositions of the present invention, because of their very low toxicity to host cells, may advantageously be employed prophylactically to prevent viral infection or to prevent the occurrence of clinical symptoms associated with the viral infection, or may be administered continuously for prolonged time to delay the onset of viral infection. Thus, the present invention also encompasses methods for the prophylactic treatment of viral infections, and in particular influenza and HSV-1 infections. In this aspect according to the present invention, the present compositions are used to prevent or delay the onset of the viral infection or related diseases in patients, including those patients who are immunodeficient or immunocompromised, for example, AIDS patients and transplant patients, among others as well as pregnant women, elderly and children.

In addition, the compositions of the present invention can be administered alone, or can be administered with additional active agent. According to certain embodiments, the additional active agents are those known to have anti-viral effects. The composition of the present invention can be administered in a single composition or in separate compositions. Administration of separate composition can be simultaneously, concomitantly, sequentially and/or alternately.

The combination of phytoene and phytofluene or a composition comprising same can be also used in methods of research purposes, particularly in methods of screening for effective anti-viral compounds. Such use is based on the characteristics activity of the phytoene and phytofluene combination, which modulate the innate cellular defense mechanism but does not affect or have a minor effect on the central immune system. Thus, according to a further aspect, the present invention provides a method for delaying viral infection of an isolated cell or tissue, comprising administering to the cell or tissue an effective amount of a combination of phytoene and phytofluene or a composition comprising same. According to certain embodiments, the isolated cell or tissue form part of a cell culture or a tissue culture. Any composition suitable for use with the combination of phytoene and phytofluene as is known to a person skilled in that art can used according to this method of the invention.

According to some embodiments, delaying viral infection of a cell or tissue of the cultures results in delaying the onset of the viral infection. According to some embodiments, delaying viral infection of a cell or tissue results in modulating at least one response against the viral infection within the cell or the tissue.

According to certain embodiments, the cellular response is selected from the group consisting of preventing and/or inhibiting virus adherence and/or penetration into the cell or tissue; inhibiting the virus replication within a cell of the subject comprising the virus; inhibiting hijacking at least one cell function by the virus in a cell comprising the virus; inhibiting the spread of the virus from a cell comprising the virus into at least one additional cell; inducing the expression of at least one gene associated with a cellular mechanism suppressing the viral infection; and inhibiting the expression of at least one gene associated with a cellular mechanism inducing the viral infection. Each possibility represents a separate embodiment of the present invention.

According some embodiments, administering the combination of phytoene and phytofluene or a composition comprising same for delaying viral infection of an isolated cell or tissue provides a positive control in determining anti-viral activity of a test compound, wherein the test compound is administered to the same type of cell or tissue under the same experimental conditions. According to some embodiments, the combination of phytoene and phytofluene or a composition comprising same is administered at an amount found to have a therapeutic effect in delaying viral infection.

According to other embodiments, the combination of phytoene and phytofluene or a composition comprising same is administered at an amount calibrated for the type of cell or tissue used.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1

### Preparation of a Composition Comprising Phytoene and Phytofluene

Fruit of proprietary tomato plants, rich in phytoene and phytofluene and essentially depleted from lycopene and zeta-carotene were used for preparing tomato oleoresin. The oleoresin was prepared by extraction with organic solvent (hexane) of the tomato fruit followed by evaporation of the organic solvent.

An exemplary resulting oleoresin contained phytoene and phytofluene (P&P) at a combined concentration of 107 mg P&P/ml and 3.04 mg β-carotene/ml oleoresin, with other carotenoids being present in negligible or undetected amounts.

The oleoresin was dissolved in DMSO to a final total phytoene and phytofluene concentration of 1-2 mM (0.75-1.0 mg P&P/ml). The composition, designated COMP1, was aliquoted to 0.1 ml samples and stored at −20° C. until usage. Leftovers after usage were discarded in order to avoid repeated freezing and thawing.

### Example 2

### Effect of a Composition Comprising Phytoene and Phytofluene (COMP1) on Gene Expression

Keratinocytes were seeded in 24-wells and cultured in culture medium (Keratinocytes SFM supplemented with EGF 0.25 ng/ml, Pituitary extract (PE) 25 μg/ml, Gentamycin 25 μg/ml) for 48 hour. The medium was then replaced by assay medium (Keratinocytes SFM supplemented with Gentamycin 25 μg/ml); the cells were incubated for 24 h. After the incubation cells were either treated with a composition comprising phytoene and phytofluene, prepared as described hereinabove (COMP1) or not (control). The composition was diluted to a final concentration of 0.05-1 μM phytoene and phytofluene and added to the cell culture medium. The cells were then incubated for additional 24 h. The control contained DMSO at 0.005%.

At the end of the incubation RNA was extracted from cells of the treated and control cells. Total RNA from each sample was extracted using TriPure Isolation Reagent according to the supplier instructions. The extracted RNA was then subjected on Affymetrix U219 gene array chip for analysis. Among 30,000 tested genes, the expression of 384 genes was found to be significantly modulated following administration of COMP1. Of the modulated genes, 12 genes, listed in Table 1, are related to antiviral activity of the cell.

TABLE 1
List of genes, the expression of which is affected by
administration of COMP1 to cultured human keratinocytes

| | | | | Expected |
|---|---|---|---|---|
| | | | | Effect of |
| Fold | Gene | Transcript ID | Exemplary activity of | Modulation of |
| Induction | Symbol | (RefSeq) | Gene | Expression |
| 2.1 | RSAD2 | NM_080657 | RSAD2 encodes for a | Induces an |
| | | | protein that is part of the | INF-mediated |
| | | | interferon (INF) | anti viral |
| | | | stimulated gene (ISG) | activity. |
| | | | complex. It is up- | |
| | | | regulated during a | |
| | | | number of viral infections | |
| | | | and is known for its | |
| | | | multifaceted anti viral | |
| | | | activity. | |
| 2.15 | IFIT1 | NM_001548 | IFIT1 gene encodes for | Innate Immune |
| | | | an interferon-induced | response (IFN |
| | | | antiviral RNA-binding | pathway) |
| | | | protein that specifically | |
| | | | binds single-stranded | |
| | | | RNA bearing a 5- | |
| | | | triphosphate group (PPP- | |
| | | | RNA), thereby acting as a | |
| | | | sensor of viral single- | |
| | | | stranded RNAs and | |
| | | | inhibiting expression of | |
| | | | viral messenger RNAs | |
| 2.3 | IFIT3 | NM_001549 | IFIT3 gene encodes for | Innate Immune |
| | | | an IFN-induced antiviral | response (IFN |
| | | | protein which acts as an | pathway) |
| | | | inhibitor of cellular as well | |
| | | | as viral processes, cell | |
| | | | migration, proliferation, | |
| | | | signaling, and viral | |
| | | | replication. | |
| 0.271 | DDX17 | NM_006386 | DDX17 gene encodes a | Reduced |
| | | | cellular RNA-binding | synthesis of |
| | | | protein involved in | viral RNA |
| | | | transcriptional regulation. | within the host |
| | | | DDX17 was shown to be | cells |
| | | | hijacked by the Influenza | |
| | | | virus for an intact viral | |
| | | | RNA synthesis | |
| 0.302 | ATF6 | NM_007348 | ATF6 gene encodes a | Defects in viral |
| | | | transcription factor that | RNA |
| | | | activates target genes for | translation, |
| | | | the unfolded protein | protein |
| | | | response (UPR) during | synthesis and |
| | | | endoplasmic reticulum | virus |
| | | | (ER) stress. This protein | production |
| | | | has been identified as a | |
| | | | survival factor that can he | |
| | | | activated by certain | |
| | | | viruses to use as a pro- | |
| | | | survival mechanism | |
| | | | required for their | |
| | | | replication. | |
| 0.283 | TOP1 | NM_003286 | TOP1 gene encodes a | Reduced viral |
| | | | protein responsible for | DNA/RNA |
| | | | the release of the | replication and |
| | | | supercoiling tension of | production of |
| | | | the DNA introduced | new viruses |
| | | | during replication and | |
| | | | transcription. It is highly | |
| | | | essential for an intact | |
| | | | cellular DNA replication | |
| | | | and transcription. TOP1 | |
| | | | was shown to be hijacked | |
| | | | by viruses such as KSHV | |
| | | | for its benefit of | |
| | | | replication and | |
| | | | propagation | |
| 0.383 | USP47 | NM_017944 | USP47 gene encodes for | Reduced |
| | | | a Ubiquitin-specific | proper viral |
| | | | protease that specifically | entry into the |
| | | | deubiquitinates | host cell |
| | | | monoubiquitinated DNA | |
| | | | polymerase beta (POLB) | |
| | | | inducing its stabilization. | |
| | | | USP47 was shown to be | |
| | | | highly essential for | |
| | | | influenza virus (PR8) | |
| | | | entry in to the host cells. | |
| 0.399 | KIF5B | NM_004521 | KIF5B gene encodes for | Reduced viral |
| | | | dynein, a microtubule- | movement |
| | | | dependent motor protein | required for |
| | | | required for normal | the |
| | | | distribution of | establishment |
| | | | mitochondria and | of infection |
| | | | lysosomes. Many | and |
| | | | different viruses utilize | replication. |
| | | | cytoplasmic dynein to | |
| | | | facilitate | |
| | | | their directed movement | |
| | | | towards the centrosomes | |
| | | | during the | |
| | | | initial establishment of | |
| | | | infection | |
| 0.423 | HTATSF1 | NM_001163280 | HTATSF gene encodes | Reduce |
| | | | for a transcription factor | synthesis of |
| | | | playing a role in the | viral RNA |
| | | | process of transcriptional | within the host |
| | | | elongation. In case of | cells |
| | | | infection by HIV-1, it is | |
| | | | up-regulated by the HIV- | |
| | | | 1 proteins and acts as a | |
| | | | cofactor required for an | |
| | | | enhanced transcription of | |
| | | | the virus. | |
| 0.436 | SRPK1 | NM_003137 | SRPK1 gene encodes for | Reduced viral |
| | | | a Serine/arginine-rich | DNA/RNA |
| | | | protein-specific kinase | replication and |
| | | | involved in the | production of |
| | | | phosphorylation of SR | new viruses |
| | | | splicing factors and the | |
| | | | regulation of cellular | |
| | | | splicing and chromatin | |
| | | | reorganization. SRPK1 | |
| | | | and 2 were shown to be | |
| | | | highly essential for the | |
| | | | replication of several | |
| | | | viruses such as Sindbis | |
| | | | virus, HIV, and | |
| | | | cytomegalovirus | |
| 0.45 | PCBP2 | NM_005016 | PCBP2 gene encodes for | Reduced virus |
| | | | a protein responsible for | DNA/RNA |
| | | | translational control and | replication and |
| | | | stability of cellular | RNA stability |
| | | | mRNAs. PCBP2 was | for translation |
| | | | shown to be essential as a | |
| | | | translational co-activator | |
| | | | of polio, papiloma and | |
| | | | hepatitis A virus RNA. | |
| 0.462 | ACBD3 | NM_022735 | ACBD3 gene encodes for | Reduced viral |
| | | | a protein that is involved | DNA/RNA |
| | | | in the maintenance of | replication and |
| | | | Golgi structure required | production of |
| | | | for the sorting and | new viruses |
| | | | modification of proteins | |
| | | | exported from the | |
| | | | endoplasmic reticulum. | |
| | | | ACBD3 was shown to be | |
| | | | essential to form a | |
| | | | replication complex for | |
| | | | picornaviruses required | |
| | | | for their intact replication | |
| | | | and propagation. | |

The expression of the genes RSAD2, IFIT1 and IFIT3 was elevated following administration of phytoene and phytofluene. These genes are involved, among other pathways in the INF-Gamma pathway of the innate immune response. This pathway is known to be activated during viral infection as part of a defense mechanism of the infected cell. Without wishing to be bound by any specific theory or mechanism of action, the elevated expression of these genes following administration of phytoene and phytofluene is expected to enhance defense mechanisms against viral infection.

The expression of the rest of the genes listed in Table 1 was attenuated. These genes are involved in cellular pathways usually utilized by viruses for their penetration, replication and propagation in the host cell. Without wishing to be bound by any specific theory r mechanism of action, by attenuating the expression level of these genes, phytoene and phytofluene may interfere with viral reproduction and spreading. One possible result of this effect may be a reduction in the outcome of a disease. In addition, the reduced expression of such cellular genes provide a second line of anti-viral protection by phytoene and phytofluene, by combating virus strains that evade the cellular innate immune response, negatively affecting the viral mechanism of reproduction and spreading.

### Example 3

### Serial Dilutions of COMP1

The threshold COMP1 dilution having cytotoxic activity was examined to determine the applicable range for examining the anti-viral effect of the composition. Series of dilutions of COMP1, standardized for concentration of phytoene and phytofluene was performed. For experiments with HSV1, the first dilution of COMP1 was 1:50 giving rise to a final concentration of 1-2% DMSO. This dilution was prepared using a medium containing 10% inactivated fetal calf serum (FCS). Then, serial double dilutions were prepared using an infection medium harboring 2% FCS producing compound dilution of 1:100 up to 1:25,600 (Table 2).

For Influenza virus, the source material was diluted 1:100 using a medium containing 10% inactivated fetal calf serum (FCS) giving rise to a final concentration of 1-2% DMSO. This source material w was diluted 1:4, and this initial dilution which proceeded with an additional 9 serial double dilutions producing a final compound dilution of 1:200, up to 1:25,600 (Table 2).

TABLE 2
Dilutions of COMP1 and final combined concentrations
of phytoene and phytofluene

| | Phytoene and phytofluene |
|---|---|
| | (Combined amount in COMP1) |
| Dilution in viral infection medium | μM |
| 1:50 | 35.4 |
| 1:100 | 17.7 |
| 1:200 | 8.85 |
| 1:400 | 4.4 |
| 1:800 | 2.2 |
| 1:1,600 | 1.1 |
| 1:3,200 | 0.55 |
| 1:6,400 | 0.28 |
| 1:12,800 | 0.14 |
| 1:25,600 | 0.07 |

### Example 4

### Cytotoxicity of COMP1

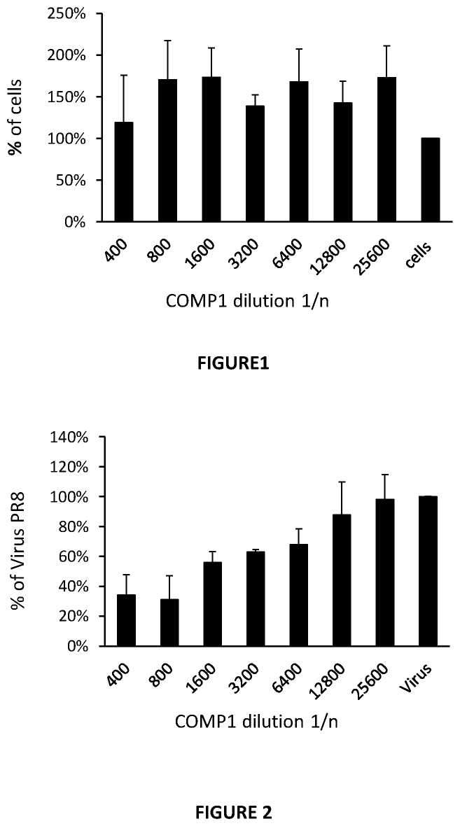

Human Madin-Darby Canine Kidney Epithelial Cells (MDCK cells) were cultured in a 96-well plate. Serial double dilutions of COMP1, in the range of 1:400 to 1:25,600 for influenza virus and 1:100 to 1:25,600 for HSV-1 (Table 2) were applied to the cultured cells in triplicates. Cells treated with similar DMSO dilutions, as well as untreated cells, served as negative controls. After a three days incubation period, the cells were fixed, and dyed with 1% gensin violet. Dye intensity was detected as O.D (Optical Density) using a spectrophotometer at a wavelength of 595 nm. Lack of cytotoxicity was indicated by a similar O.D values observed in the untreated control compare to the different dilutions of COMP1. COMP1 did not have a cytotoxic effect on the cultured cells as tested under the mentioned above serial dilutions (FIG. 1). Compound dilution of 1:400 was the first dilution to be tested for anti viral activity (1:100, 17.7 μM or 10 μg/ml phytoene and phytofluene).

Example 5: Effect of COMP1 on influenza virus propagation in cultured cells

MDCK cells were seeded in a 96-well plate (10,000 cells/well) in 100 μl DMEM medium containing 2% FCS. The cells were incubated for at least 5 hours at a temperature of 37° C., 5% CO₂. After the first incubation time, COMP1 prepared and diluted as described in Example 3 and Table 2 hereinabove was added, and the cells were incubated for 24 h (dilution 1:400 up to 1:25,600). Cells were then infected with a PR8N1H1 strain of influenza virus diluted 1:2,000 (final concentration 50 plaque forming units (PFU) per well) and incubated with the virus for additional 3 days in the presence of COMP1. In parallel, a standalone virus infection was carried out to evaluate the efficiency of the infection and the maximal virus reproduction for comparison. Virus particles were determined using an ELISA assay against envelope proteins of the virus giving a quantitative representation for the amount of viruses at the end point of the experiment.

FIG. 21 is a graph showing inhibition of the particle number of influenza virus (N1H1 PR8) within NDCK cells by COMP1 (dilution 1:400 up to 1:25,600 of Table 2). As shown in FIG. 1, a composition comprising a combination of phytoene and phytofluene prepared as described in Example 1 hereinabove (COMP1) inhibited the reproduction of influenza virus in a dose responsive manner, reaching above 60% inhibition under a phytoene and phytofluene concentration of 4.4 μM (dilution 1:400). No cytotoxic activity against MDCK cells was observed for any of the examined dilutions of phytoene and phytofluene (FIG. 2). Similar results were also obtained when MDCK cells were infected with additional influenza strain (H3N2) (data not shown).

### Example 6

### COMP1 effect on propagation of HSV1 in cultured cells

Human kidney (HuKi) cells were seeded in a MEM HAA medium containing 10% FCS. COMP1 at several dilutions of 1:200, 1:400 and 1:800 (obtaining a final phytoene and phytofluene concentration as indicated in Table 2) was added to the cells. Next, cells were infected with HSV1 (vr-3-p8) at 0.1MOI (Multiplicity of infection) in the presence of COMP1 for a period of 1 h which was terminated by washing out the virus. The infected cells were then incubated in the presence of COMP1 for additional 72 h. Supernatants of the infected cells were then collected, decimal serial diluted to obtain several viral titers and used for a second infection experiment. Second infection was performed for a 72 h ending with the formation of plaques, where cells exploded following virus exit. A Semi-quantification method was then applied in order to determined plaque number in the presence or absence of COMP1. Cells were fixed with 20% ethanol, stained with 1% gensin violet and pictured for demonstration. Acyclovir known as an effective HSV1 drug served as a positive control during the experiment.

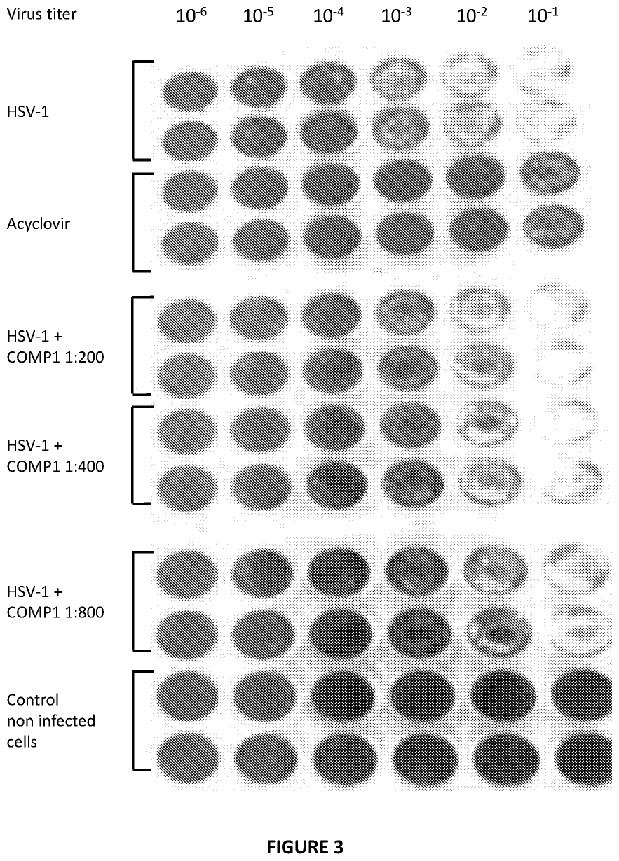

FIG. 3 is a gensin violet staining of the infected cells demonstrating an inhibitory effect of COMP1 on HSV1 reproduction. As indicated in the figure, fewer plaques (white dots) can be observed in all tested dilutions (1:200, 1:400 and 1:800) when compared to cells infected with virus alone. This plaque reduction was determined in all measured virus titers. In order to further quantify the effect of this virus, load obtained at each compound dilution was collected and used for a plaque assay. One day prior to infection, HK cells (3*105cells/well in a 24 well plate) were seeded in M199 medium supplemented with 10% heat-inactivated FBS. The cells were infected with HSV 1 at MOI of 0.1 (stock virus titer : 10⁸*5.5 PFU/ml). The virus was allowed to be adsorbed for 1 h with constant shaking, and was then removed. The cells were washed twice with M199 medium and then overlaid with M199 medium supplemented with 2% FBS alone or with the following stock dilution of COMP1: 1:200, 1:400, and 1:800. Viral yields were determined48 hours post infection, by titration of the supernatants on Vero cells using plaque assay. The formed plaques were then counted and the counting values were then used for quantifying the inhibitory effect of COMP1. As indicated in FIG. 3, treatment with COMP1 , regardless of the dilution used , caused approximately 50% reduction in virus yield. It is most likely that elevating phytoene and phytofluene concentration above 4.4 μM will increase their inhibitory effect on HSV1 reproduction No cytotoxic activity against HuKi cells was observed for any of the indicated dilutions (data not shown).

### Example 7

### COMP1 effect on in vivo propagation of influenza virus in mice

COMP1 prepared as described in Example 1 hereinabove was used in the experiment. Female BALV/c mice (age of 5 weeks at a weight of 14-16 g) were used. 50 μ1 of COMP1 at a 1:50 dilution (35.4 μM as in Table 2 above) were given to each mouse by drop-wise nasal administration 8 hours before infection with influenza virus PR8 N1H1. After 8 hours, mice were anesthetized using isoflurane and 50 μ1 of the virus (40PFU per mouse) were administered drop wise into the mice nose.

The assay included three groups:
1. Mice infected with the influenza virus with no additional treatment (“sick mice”).
2. Mice treated with COMP1 as described above.
3. Mice treated with oseltamivir phosphate (“Tamiflu”) at the same protocol as mice treated with COMP1.
5 days after infection the mice were again anesthetized and the lungs of each mouse were harvested and stored at −80 ° C. until used. For determining the virus concentration within the lungs, the lungs from each mouse were homogenized using SPEX balls in 1 ml DMEM without FCS. 100 μl of the obtained suspension were lysed and polynucleotides were isolated using Easy Mag according to the manufacturer protocol. [To the inventors: please provide manufacturer for both homogenization and lysis protocol] Real-time (RT) PCR was performed with the isolated polynucleotides using specific primers for influenza hemagglutinin encoding gene using TaqMan. The threshold cycle (Ct) values obtained were transformed to virus concentration (copies/ml) using pre-determined calibration curve.

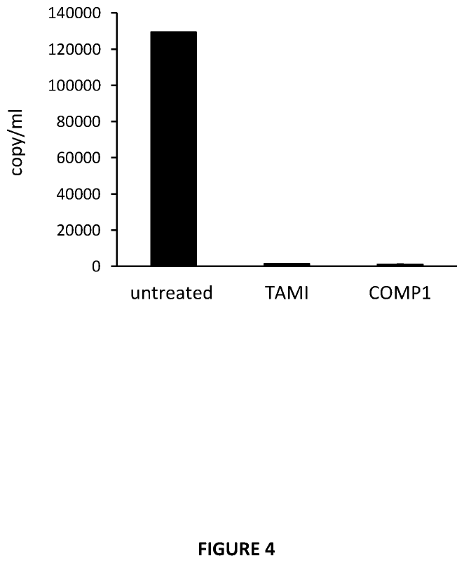

As is clearly demonstrated in FIG. 4, the inhibitory effect of COMP1, comprising a combination of phytoene and phytofluene at a concentration of 35.4 μM (about 20 μg/ml) inhibited in vivo replication of the influenza virus PR8 at the same extent as oseltamivir phosphate, known to be highly effective in preventing and treating influenza symptoms. This is the first time that such effect of phytoene and phytofluene is demonstrated.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-36. (canceled)

37. A method for treating a viral infection of epithelial cells of a subject's mouth, the method comprising topically administering to the epithelial cells an effective amount of a combination of phytoene and phytofluene, wherein the effective amount of the combination of phytoene and phytofluene is about 2.2 thereby treating the viral infection.

38. The method of claim 37, wherein the subject is a human.

39. The method of claim 37, wherein the viral infection is caused by a herpes simplex virus-1 (HSV-1) or an influenza virus.

40. The method of claim 37, wherein the effective amount of a combination of phytoene and phytofluene is topically administered in a composition comprising the combination of phytoene and phytofluene and an excipient, diluent, or carrier.

41. The method of claim 40, wherein the composition further comprises at least one additional ingredient comprising an amino acid, a polypeptide, a peptide, a fatty acid, a fat, a sugar compound, or a carotenoid, wherein the carotenoid is other than phytoene and phytofluene or a combination thereof

42. The method of claim 41, wherein the carotenoid that is other than phytoene and phytofluene is a carotenoid of the xanthophylls group or a non-pro-Vitamin A carotenoid.

43. The method of claim 37, wherein the effective amount of the combination of phytoene and phytofluene is between 1.1 μM and 2.2 μM.

44. A method for delaying viral infection of epithelial cells, the method comprising administering to the epithelial cells an effective amount of a combination of phytoene and phytofluene, wherein the effective amount of the combination of phytoene and phytofluene is at least 0.05 thereby delaying the viral infection.

45. The method of claim 44, wherein the subject is a human.

46. The method of claim 44, wherein the viral infection is caused by a herpes simplex virus-1 (HSV-1) or an influenza virus.

47. The method of claim 44, wherein the effective amount of a combination of phytoene and phytofluene is topically administered in a composition comprising the combination of phytoene and phytofluene and an excipient, diluent, or carrier.

48. The method of claim 47, wherein the composition further comprises at least one additional ingredient comprising an amino acid, a polypeptide, a peptide, a fatty acid, a fat, a sugar compound, or a carotenoid, wherein the carotenoid is other than phytoene and phytofluene or a combination thereof.

49. The method of claim 48, wherein the carotenoid that is other than phytoene and phytofluene is a carotenoid of the xanthophylls group or a non-pro-Vitamin A carotenoid.

50. The method of claim 44, wherein the effective amount of the combination of phytoene and phytofluene is between 1.1 μM and 2.2 μM.

51. A method for treating a viral infection of epithelial cells, the method comprising administering to the epithelial cells an effective amount of a combination of phytoene and phytofluene, wherein the effective amount of the combination of phytoene and phytofluene is about 2.2 μM, thereby delaying the viral infection.

52. The method of claim 51, wherein the epithelial cells are maintained in tissue culture.

53. The method of claim 51, wherein the combination of phytoene and phytofluene is administered in a composition.

54. The method of claim 51, wherein the administering the combination of phytoene and phytofluene or a composition comprising same for delaying viral infection of the epithelial cells provides a positive control in determining anti-viral activity of a test compound, wherein the test compound is administered to the same type of cell or tissue under the same experimental conditions.
